SEQUENCE LISTING (2 sequences)

SEQ ID NO: 1 - DNA, 19 bp, Artificial Sequence
  Sequence found in microbes
gtgycagcmgccgcggtaa

SEQ ID NO: 2 - DNA, 20 bp, Artificial Sequence
  Sequence found in microbes
  misc_feature (8)..(8): n is a, c, g, or t
ggactacnvgggtwtctaat

(19) United States
(12) Patent Application Publication
(10) Pub. No.: US 2018/0320221 A1
(43) Pub. Date: Nov. 8, 2018

(54) METHOD OF IDENTIFYING AND QUANTIFYING BENEFICIAL AND HARMFUL MICROBES IN WASTEWATER TREATMENT PROCESSES

(71) Applicant: MICROBE DETECTIVES LLC, Downers Grove, IL (US)

(72) Inventor: Trevor Wayne GHYLIN, Wauwatosa, WI (US)

(21) Appl. No.: 15/961,816

(22) Filed: Apr. 24, 2018

Related U.S. Application Data
(60) Provisional application No. 62/488,918, filed on Apr. 24, 2017.
(60) Provisional application No. 62/488,913, filed on Apr. 24, 2017.
(60) Provisional application No. 62/501,857, filed on May 5, 2017.

(51) Int. Cl. C12Q 1/689; C12Q 1/04; C12Q 1/6806

(52) U.S. Cl. CPC C12Q 1/689; C12Q 1/6806; C12Q 1/04

(57) ABSTRACT

Embodiments of the present invention relate to a method of detecting, identifying, and quantifying microbes in a wastewater treatment process or facility. The invention identifies the microbes by manipulation of their DNA. Microbes are collected, and a portion of the DNA sequence of a particular gene that is found in microbes is amplified. The amplified DNA sequence contains a highly variable region that can be used identify the microbe. Each amplified DNA sequence is matched against known sequences in various microbes. The identity of the microbes present in a sample can be determined and, sometimes, their biological functions too. Also, the relative quantities of the microbes, compared to the total microbial pool, can be determined. With this information, harmful microbes can be identified and targeted for removal from the wastewater, and beneficial microbes can be cultivated as desired.

### CROSS-REFERENCE TO RELATED APPLICATIONS

This application claims priority to and benefit of Provisional Application No. 62/488,913 filed on Apr. 24, 2017; Provisional Application No. 62/488,918 filed on Apr. 24, 2017; and Provisional Application No. 62/501,857 filed on May 5, 2017, all of which are hereby incorporated by reference herein in their entirety.

### FIELD OF THE INVENTION

An embodiment of the present invention relates to a method of detecting, identifying, and quantifying microbes in a wastewater treatment distribution process and, more particularly, to a method of identifying and quantifying the microbes via manipulation of their DNA.

### BACKGROUND OF THE INVENTION

An embodiment of the present invention relates to a method of detecting, identifying, and quantifying microbes in a wastewater treatment distribution process and, more particularly, to a method of identifying and quantifying the microbes via manipulation of their DNA.

Wastewater treatment includes many biological processes that are performed by microbes. Wastewater treatment facilities maintain a suitable environment that allows the microbes' natural processes to break down pollutants under controlled conditions. The microorganisms also break down and remove many of the nutrients and organic matter in the wastewater. Microbes are the primary workhorses in wastewater treatment plants and they perform a wide variety of biological processes.

Some microorganisms convert organic compounds, such as fats, sugars, proteins, and minerals, such as phosphorus, nitrogen, iron, potassium, and calcium, into materials that are beneficial for the environment. Specific microbes such as ammonia/nitrite oxidizing bacteria/archaea, phosphorus accumulating organisms, denitrifying microbes, fermenters, methane producers and others are responsible for converting soluble pollutants such as organic carbon, nitrogen, and phosphorus, into particulate or gas form ultimately removing them from the water. In addition to clearing organic compounds from the water, the microbes can also convert certain organic wastes into forms for easy removal from the wastewater.

Wastewater treatment systems must maintain the right environmental conditions to foster the microorganisms suited for processing the wastewater. The presence of the wrong microbes can interfere with the various processes. Non-beneficial microbes such as filaments, foamers, biofilm formers and slime producers are nuisance organisms and cause problems in the treatment system. Other microbes can be pathogenic and cause sickness or disease.

In wastewater treatment systems, the microbes are usually grown and maintained as a biomass, or a mixture of living or dead microorganisms. The precise composition of a biomass can fluctuate over time and can vary as environmental conditions change. Because a biomass is maintained in an environment designed to foster microbial life in general, any foreign microbe that enters the system can thrive and disrupt the normal biological processes of the biomass. However, in order to deal with harmful microorganisms, they must first be detected and identified.

Because of the physical complexity of a biomass and because it can contain many different microorganisms, it is nearly impossible to detect, identify and quantify the beneficial and nuisance microbes present in wastewater treatment plants. Many wastewater operators have no idea what microbes are present in their systems.

If there is a serious problem in the wastewater treatment processes (for example, poor settling leading to effluent solids permit violation), a sample of the biomass can be sent to an expert for visual or microscopic inspection. Microscopic inspection can reveal the identity of one or a handful of bacteria based on morphotype; that is, the shape and size of a bacteria and its ability to absorb different stains.

This process is time-consuming and relies on a small number of experts to make subjective judgments as to the identity of the microbes. As the vast majority of microbes (>99%) in the biomass cannot be visually identified or quantified using a microscope, very little information is ultimately obtained from this process. Furthermore, it relies on an analysis of a portion of the biomass which might not be truly representative of the entire biomass.

Even where conventional tests can detect the presence of microbes, those microbes can be present in quantities too small to identify the exact kinds of microbes that are present. In mixed samples, microbes present in greater abundance can mask the presence of microbes present in lesser quantities. It can be particularly difficult to identify the microbes that are present in mixed, heterogeneous combinations.

Many wastewater distribution systems contain features that interfere with certain alternative microbial detection methods. For example, microbes can be grown in culture until they multiply into sufficient numbers for detection and analysis, though this method requires sufficient time for several generations of microbial reproduction. However, wastewater distribution systems can include additives or preservatives that inhibit or suppress certain bacteria or microbes from growing in these types of water quality tests. Water quality monitoring based on the culturing of the microbes cannot detect contaminant microbes under these conditions. Alternatively, significant amounts of contamination can go undetected because the additive in the water suppresses the bacteria's ability to grow in culture.

There is a particular need to monitor the composition of the microbes in a wastewater treatment facility, to ensure that there is an optimal or desired mixture of microbes to process the different elements in the wastewater as desired. There is a need to determine whether desired microbes are decreasing in number or gone altogether. There is a need to monitor for the appearance of harmful microbes that can adversely affect the beneficial microbes, impede normal treatment processes, or cause disease. There is also an ongoing need to monitor whether the relative amounts of beneficial microbes are present is desired proportions for optimal or desired performance.

As can be seen, there is a need for an improved method of detecting, identifying, and quantifying the microbes present in wastewater treatment processes.

### SUMMARY OF THE INVENTION

An embodiment of the present invention relates generally to a method of detecting and identifying a variety of microbes present in a wastewater treatment process, as well as determining if certain microbes are absent from the system. The invention also relates to determining the relative quantities of the different microbes, or families of related microbes, or groups of microbes that perform similar biological functions. In this way, the presence, identity, and quantity of beneficial and harmful microbes can be determined.

One aspect of the invention relates to a method of collecting samples from the wastewater facility and manipulating the DNA of the microbes present in the sample. This method targets the DNA of genes that are commonly found in most microbes, but that also contain a hypervariable region with a DNA sequence unique to each species, genus, family, order, class, or phylum of microbes.

Generally, the invention provides a method of monitoring, detecting, identifying, and quantifying the microbes in the wastewater treatment process by the steps of: collecting samples from at least one location in the treatment plant; concentrating the biomass on a filter or other support; extracting the DNA of the microbes collected; sequencing the extracted DNA; and using the DNA sequences to identify the microbes present in the sample.

The DNA of each pertinent microbe is collected and then amplified to sufficient quantities to determine the unique sequence of the hypervariable region of each microbe—and thus to provide the identity of each pertinent microbe. This allows the identification of multiple different microbes in the same sample and can provide relative quantities of each microbe or family of microbes.

In addition to identifying individual microbial components, this information can be analyzed and processed to determine the overall composition of a biomass in terms of microbe genus or family, pathogenicity, or by functionality. For example, the relative amounts microbes that perform certain functions can be determined, such as microbes that oxidize ammonia or nitrite compounds, microbes that perform anaerobic ammonium oxidation (anammox), nitrification in one step instead of two steps (comammox), metabolize or concentrate or clear minerals (such as phosphorus, calcium, sodium, or iron), or generate methane. It can be determined whether harmful microbes are present, such as those that can create filamentous growths, form slime or biofilms, or cause disease. It is to be noted that some microbes can be classified as either beneficial or harmful (or both), depending on the particular type of wastes being treated.

The composition of a wastewater treatment biomass can be monitored over time for management, maintenance, and optimization of the microbiome of the system. Again, while certain microbes can be problematic or beneficial in isolation, the overall composition of the entire microbial system can require a precise (or imprecise) balance of particular microbes.

At least one embodiment of the present invention is able to detect microbes that are dead or that fail to grow using cell culture techniques because the detection and identification techniques are based on the presence of DNA, instead of requiring viable cells that are capable of reproduction. The invention can thus identify microbial contaminants, or beneficial or harmful microbes, even if the cells are dead or degraded, as long as the cells' DNA is sufficiently intact. Such embodiments include the sensitive detection and identification of microbes, regardless of whether the cells are viable or not viable when the samples are collected and when the analysis is performed. Therefore the present invention can detect contamination that is likely undetectable with traditional culture based water quality tests.

Further, the detection method can be tailored to determine the composition of a biomass over time and for predicting the efficiency of different functional features of the biomass, such as its ability to metabolize or process a specific molecule or compound. Likewise, a biomass can be thus monitored to assess its overall health.

An embodiment of the invention can also identify the incursion of microbes that are harmful to the biomass or adversely affect its biological processes. An embodiment of invention can also be used to assess the response of the wastewater treatment system to changes in its environment or to the addition of exogenous microbes.

An aspect of this invention relates to a kit for collecting samples from water distribution systems, the kit providing a means of obtaining a sample and transporting the sample for further DNA analysis and identification of the microbes.

### BRIEF DESCRIPTION OF THE SEQUENCE LISTING

The invention can be more fully understood from the following detailed description and the accompanying Sequence Listing, which form a part of this application.

The sequence descriptions summarize the Sequence Listing attached hereto. The Sequence Listing contains standard symbols and format used for nucleotide sequence data comply with the rules set forth in 37 C.F.R. § 1.822.

### BRIEF DESCRIPTION OF THE DRAWINGS

The features of embodiments of the present invention which are believed to be novel are set forth with particularity in the appended claims. The drawings may not be to scale. The invention can best be understood by reference to the following description taken in conjunction with the accompanying drawings.

FIG. 1 provides a chart depicting the steps of detecting, identifying, and quantifying microbes in a wastewater treatment facility.

FIG. 2 provides a chart depicting the steps of detecting, identifying, and quantifying microbes in a wastewater treatment facility, and shows an exemplary kit including the components for collecting samples for microbial analysis, the kit containing a syringe with filter attachment, sterile filter, desiccant card, processing form, and set of instructions.

FIGS. 3A-3D show the identification of microbes in a sample; FIG. 3A shows a representation of a portion of a double-stranded DNA helix; FIG. 3B shows a representation of the complementary deoxyribosenucleotide sequences of a portion of double-stranded DNA; FIG. 3C shows the identity and relative expression levels of microbes detected in several samples; FIG. 3D shows the relative composition of different types of microbes in a sample.

FIG. 4 shows a partial list of microbes that can be identified.

FIG. 5 shows the types and relative quantities of microbes related to nitrogen removal that were present in a wastewater treatment facility over time.

FIG. 6 shows the types and relative quantities of filamentous microbes detected in a wastewater treatment facility over time.

FIG. 7 shows the types and relative quantities of microbes that were identified in a wastewater treatment facility over time.

FIG. 8 shows a kit including the components for collecting samples for microbial analysis.

FIG. 9 is a pie chart depicting an example of the microbial DNA found in sample.

FIG. 10 is a pie chart depicting an example of the microbial DNA found in sample.

FIG. 11 is a pie chart depicting an example of the microbial DNA found in a sample.

FIG. 12 is a pie chart depicting an example of the microbial DNA found in a sample.

FIG. 13 is a pie chart depicting an example of the microbial DNA found in a sample.

FIG. 14 is a pie chart depicting an example of the microbial DNA found in a sample.

FIG. 15 is a pie chart depicting an example of the microbial DNA found in a sample.

FIG. 16 is a pie chart depicting an example of the microbial DNA found in a sample.

FIG. 17 is a pie chart depicting an example of the microbial DNA found in a sample.

FIG. 18 is an example of a bacterial abundance chart.

FIGS. 19A-19E is an example are examples of a bacterial relative abundance table.

### DETAILED DESCRIPTION OF THE INVENTION

While the present invention is susceptible of embodiments of various forms, there is shown in the drawings, and will hereinafter be described some exemplary and non-limiting embodiments, with the understanding that the present disclosure is to be considered an exemplification of the invention. It is not intended to limit the invention to the specific embodiments listed.

A microbe is any noncellular or unicellular (including colonial) microorganism. Microbes include all prokaryotes and eucaryotes and include bacteria (including cyanobacteria), Archaea (including sulfate-reducing Archaea), spores, lichens, fungi, molds, protozoa, virinos, viroids, viruses, phages, and some algae. As used herein, the term “microbe” is synonymous with microorganism.

Examples of microbes include Salmonella, E. coli, Enterococcus, cyanobacteria, human-associated bacteria. Genera of interest (but not limited to) are Nitrosomonas, Nitrospira, Nitrotoga, Kueninia, Anammoxoglobus, Methanosarcina, Microthrix, Gordonia, Zoogloaea, Dechloromonas, Tetrasphaera, Accumulibacter. The microbe or pathogen can be selected from a group containing of Escherichia coli, enterohemorrhagic Escherichia coli, enterotoxigenic Escherichia coli, enteroinvasive Escherichia coli, enterpathogenic Escherichia coli, Salmonella, Listeria, Yersinis, Campylobacter, Clostridial species, Staphylococcus.; frank and opportunistic bacterial, fungal, viral, parasitic pathogens; indicator organisms including heterotrophes, generic E. coli, total and fecal coliforms and enterococcus; spoilage organisms including Pseudomonas; indicator molecules including glial fibrillary acid protein (GFAP), transmissable spongiform encephalopathy (TSE) agents (prions), including bovine spongiform encephalopathy (BSE) agents, scrapie, and chronic wasting disease.

A biomass refers a mixture of microbes or bacteria. In a wastewater facility, the microbes in the biomass can be maintained on a physical support and can incorporate nutrients for feeding the microbes.

DNA, or deoxyribonucleic acid, is a self-replicating material present in most living organisms, including microbes. It provides the genetic instructions for the growth, functioning, and reproduction of living organisms, including microbes.

PCR, or polymerase chain reaction, refers to a technique used in molecular biology to amplify a single copy or a few copies of a segment of DNA in amounts up to several orders of magnitude. The invention provides PCR-based methods for accurate, quantitative measurement of both the amount of DNA present for a given indicator gene and levels of expression for the effector gene. Such measurements provide specific information on the amount of specific microorganisms present in a given ecosystem (e.g., compared to the level of a control or indicator gene), as well as specific information on the level of microorganism activity (for example, as reflected by the level of expression of an effector functional gene).

QIIME is an open-source bioinformatics tool for performing analysis of raw microbial DNA sequences, to determine the identity of the microbe the DNA was obtained from.

RNA, or ribonucleic acid, is essential in various biological roles in coding, decoding, regulation, and expression of genes.

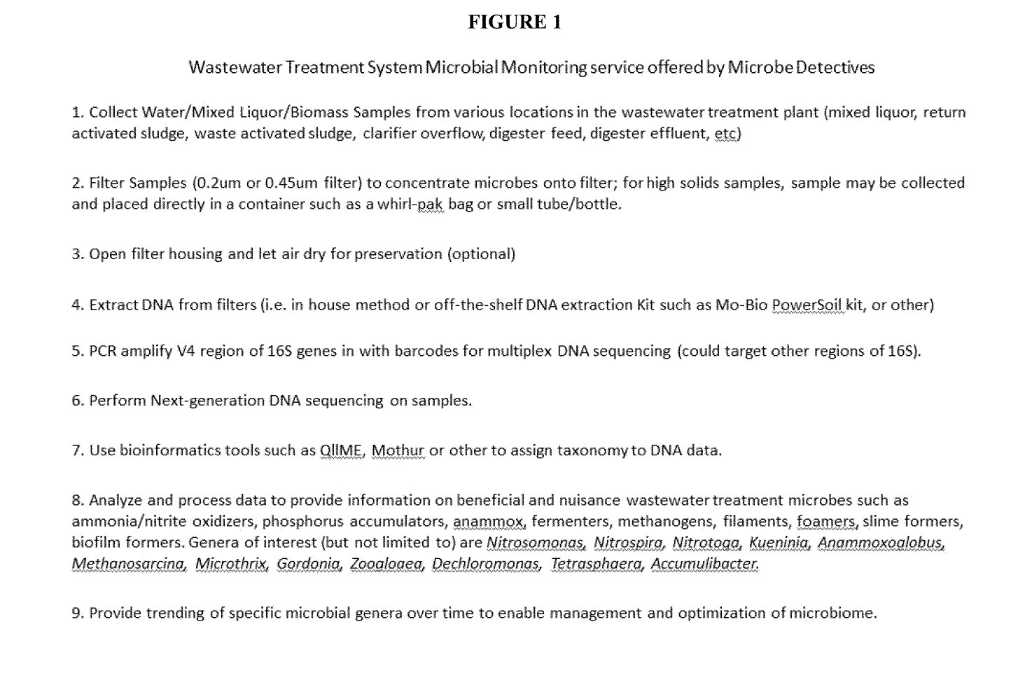

As shown in FIGS. 1-8, an embodiment of the present invention provides a method of detecting, identifying, and quantifying beneficial and harmful microbes in wastewater treatment processes. Broadly, the invention provides a method of monitoring, detecting, identifying, and quantifying the microbes in the wastewater treatment process by the steps of: collecting samples from at least one location in the treatment plant; concentrating the microbes of the sample of the biomass on a filter or other support; extracting the DNA of the microbes collected; sequencing the extracted DNA; and using the DNA sequences to identify the microbes present in the sample.

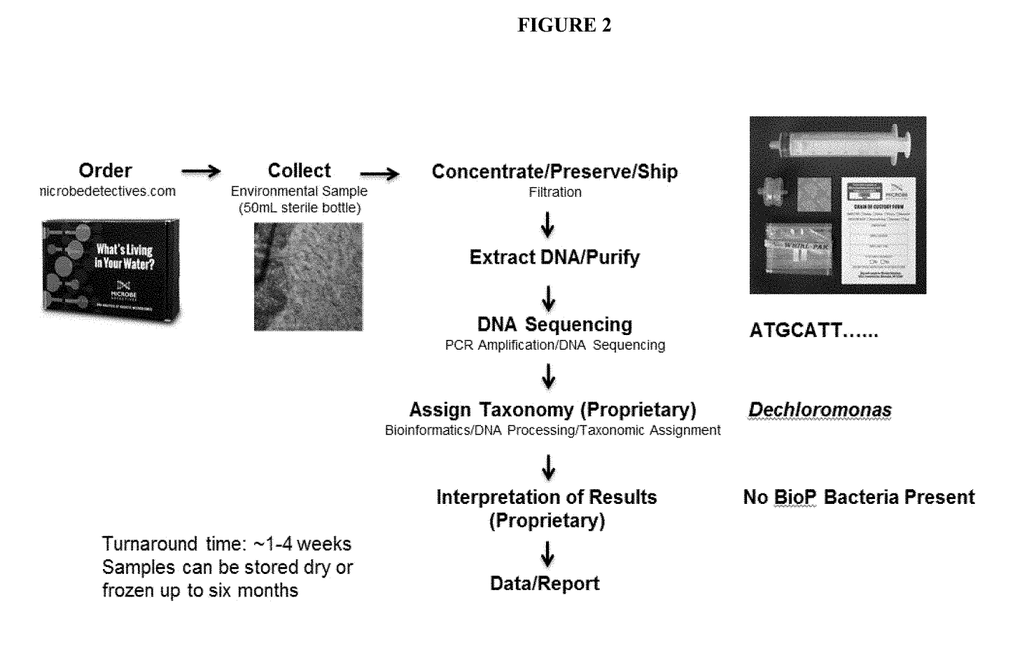

Referring now to FIGS. 1-2, an embodiment of the invention can include the following steps:
1. Collect mixed liquor or other liquid samples from one or more locations in the wastewater treatment facility. It is preferred to collect samples from locations where the biomass is located.
2. Concentrate the microbes into a compact mass and onto a support amenable for use in DNA or molecular biology technologies. For example, the mixed liquor sample can be passed through a filter having a pore size small enough to capture microbes, while allowing water to pass through. For most microbes, a filter with a 0.22 um pore can be used to separate the microbes from the sample.
3. Extract the DNA from the collected microbes. For example, a DNA extraction kit (such as DNEasy PowerSoil kit (Qiagen)) can be used to extract the DNA, according to the manufacturer's instructions. Alternatively, the DNA can be extracted using standard DNA extraction methods. See T. Maniatis, E. F. Fritsch & J. Sambrook, Molecular Cloning: A Laboratory Manual (Cold Springs Harbor Laboratory 1982).
4. Perform PCR amplification on the extracted DNA samples to amplify a hypervariable region in a gene commonly found in microbes, for example, region V3, V4, V5, and/or V6 of the bacterial 16S ribosomal RNA (rRNA) gene.
5. Perform DNA sequencing on the PCR-amplified DNA samples. This can be outsourced to a commercial vendor or done using standard molecular biology techniques. See T. Maniatis, E. F. Fritsch & J. Sambrook, Molecular Cloning: A Laboratory Manual (Cold Springs Harbor Laboratory 1982).
6. Use bioinformatics tools such as QIIME, an open-source bioinformatics pipeline for performing microbiome sequence analysis from raw DNA sequencing data, and Mothur, another open source software package for bioinformatics data processing, particularly in the analysis of DNA from uncultured microbes, to assign taxonomy and identify microbes in the samples, and to determine the relative quantities of each microbe as a percentage of the sample (or as a quantitative amount).
7. Analyze data for presence of microbes either expected to be found or alternatively not typically found in wastewater treatment systems.
8. Perform more focused sampling efforts based on locations and types of microbes revealed from previous analyses if needed, or begin investigation near sites testing positive for certain microbes.

The data can be compiled over time and analyzed to monitor the microbiome of the system. The same site(s) can be sampled and analyzed daily, weekly, bi-weekly, or monthly, or other schedule. Routine and regular sampling of the wastewater treatment facility can support analysis to identify trends in the presence of various microbial species to facilitate the management of beneficial and nuisance populations.

In various aspects, the present embodiment provides novel methods for remote testing of one or more pathogens or other microbes in food, water, wastewater, sludge, pharmaceutical, industrial samples, and the like. In particular aspects, a dry-enrichment or a semi-dry-enrichment process allows for incubation, during transit to a remote testing location, of the food samples either without (e.g., liquid samples) the addition of enrichment media, or with (e.g., solid or semi-solid samples) addition of only relatively small quantities of media and/or supplements, for testing at the remote location of contaminating pathogens or other microbes.

The samples can be obtained from one or more sites in the wastewater treatment system, and at one or more points in time, as desired. Depending on the amount of liquid available and the microbial load, typical samples can range in volume from a few microliters to a few milliliters to several liters.

The samples can be obtained from water, mixed liquor, or biomass samples from various locations throughout the wastewater treatment plant. Samples can be collected at various locations, can include (but are not limited to) mixed liquor, biofilm from media, granules, return activated sludge, waste activated sludge, clarifier overflow, secondary effluent, plant effluent, digester feed, digester effluent, and biosolids.

It is preferred that that samples be concentrated before subsequent DNA analysis, that the microbes be collected in a small volume. For example, the microbes can be collected onto a filter or membrane. It is preferred that the microbes be collected on a filter with a pore size smaller than a typical microbe (for example, having pores of 2.0 um or smaller, or preferably 0.45 um or smaller, or more preferably having pores of 0.22 um or smaller). Liquid samples can be directly applied to such filters, or applied via reverse osmotic pressure; where the samples are solid or semi-solid, they can be directly applied to such filters or supports. Alternatively, microbial samples can be collected by other techniques, such as by centrifugation.

It is envisioned that these samples can be shipped to a remote location for further processing. In such situations, the sample can be stored and shipped off-site. Before these samples are shipped, they can be fixed by standard methods, for example using alcohol or other preservatives. Otherwise, they can be shipped without fixation or preservation, with the sample processing continued at a later time or at a different site.

After the microbes have been applied to the filters, the DNA of those microbes can be extracted from the filter with standard DNA extraction techniques or technologies. For example, the DNEasy PowerSoil Kit (Qiagen) can be used, according to the manufacturer's instructions, to isolate high quality DNA from such samples in a short period of time (for example, about 30 minutes). There are a number of commercially available kits that can be used to extract DNA from a solid substrate or platform, or from a concentrated sample of microbes.

After the microbial DNA has been extracted from the filter (or other DNA collection platform), the microbial DNA can be amplified or multiplied with commonly-known molecular biology techniques and commercially-available products according to the manufacturers' instructions. See T. Maniatis, E. F. Fritsch & J. Sambrook, Molecular Cloning: A Laboratory Manual (Cold Springs Harbor Laboratory 1982).

Microbes or bacteria contain certain genes in common. For example, microbes commonly contain a 16S rRNA gene; however, that gene contains highly variable regions encoded by sequences of DNA unique to each species or family of microbes. This variation in the DNA sequence of the gene is widely used to identify the species of an individual microbe. The sequence of the 16S rRNA gene and its hypervariable regions are known for many organisms, and those sequences are available on many databases such as Greengenes and the Ribosomal Database Project.

The prokaryotic 16S ribosomal RNA gene (16S rRNA) contains nine variable regions. The variable regions are often used to identify the genus or species of an individual microbe. For example, variable region V4 of the 16S rRNA gene can be amplified using the MiSeq System (Illumina), and using primers 515F and 806R can be used to amplify the V4 region of the microbial 16s RNA gene. In most species, the fourth hypervariable (V4) region can be analyzed to identify that microbial species.

For primer 515F (SEQ ID: 1), the sequence is: GTGYCAGCMGCCGCGGTAA.

For primer 806RB (SEQ ID: 2), the sequence is: GGACTACNVGGGTWTCTAAT.

These primers can be used in standard PCR conditions to amplify the variable region V4 of the 16S rRNA gene. Other primers are commercially available for amplifying the V4 region of the 16S rRNA gene. Other primers are routinely used to amplify other hypervariable regions of the 16S rRNA gene (including but not limited to regions V3, V4, V5, and/or V6 of the bacterial 16S rRNA gene).

The amplified microbial DNA, such as amplified regions of the V4 region of the 16S rRNA gene can then be sequenced by commonly-known molecular biology techniques and commercially-available products according to the manufacturers' instructions. Alternatively, this process can be outsourced to certain commercial vendors. The 16S rRNA gene is a widely used gene marker for genus and species identification and taxonomic significance in bacteria and archaea. The estimated substitution rate for hypervariable regions is thousands of times higher than for highly conserved regions; the genetic differences of these hypervariable regions provide abundant taxonomic information about microbes. Therefore, the 16S gene amplicons obtained from PCR or other molecular biology techniques can be used to make taxonomic identifications based upon bioinformatics alignments of genetic sequences.

The 16s rRNA gene is a widely used gene marker for genus and species identification and taxonomic significance in bacteria and archaea. The estimated substitution rate for hypervariable regions is thousands of times higher than for highly conserved regions; the genetic differences of these hypervariable regions provide abundant taxonomic information about microbes. Therefore, the 16S gene amplicons obtained from PCR or other molecular biology techniques can be used to make taxonomic identifications based upon bioinformatics alignments of genetic sequences.

Like 16S rRNA in microbes, 18S rRNA is commonly used for phylogenetic analyses in fungi, and it has more hypervariable domains than 16S rRNA. Also, the ITS (Internal Transcribed Spacer) region (which includes 5.8S), is deleted in the posttranscriptional process of nuclear rRNA cistron, and is commonly regarded as a universal fungi barcode marker. The ITS region is routinely used for the identification of a broad range of fungi. Compared to 18S, the ITS region has greater variability and can be more suitable as a genetic marker for measuring intraspecific genetic diversity in fungi.

Just as microbes can be identified through DNA sequencing of the 16S rRNA in microbes, so too can fungi present in the wastewater samples be detected and identified by DNA sequencing of the 18S rRNA or ITS regions.

Embodiments of the present invention relates to a DNA-based microbial analysis service to detect different microbes in a wastewater treatment system. Some embodiments can detect minute amounts of contaminating microbes even if the microbes are dead or unable to proliferate normally. Some embodiments can also decipher the source of contamination, as many microbes are associated with very specific sources (for example, salmonella and enterococcus avium are indicative of contamination from birds, especially chickens). Other microbes are associated with particular plants or animals, particular industries, or particular agricultural or industrial processes.

Some embodiments include water sampling and analysis of microbial data to determine the health of the biomass. The other steps of the embodiments can be changed based on other methods for microbial detection and identification, especially as technology changes. Future methods can use RNA sequencing or other technology in lieu of DNA sequencing, in order to determine the identity and/or quantity of individual microbes. Future methods can also incorporate methods for removing nonviable DNA, such as binding extracellular DNA with propidium monoazide prior to sequencing (or other methods as they become available).

Presently, there are many commonly-used methods for sequencing the DNA of collected samples. For example, the present embodiment can be practiced by traditional polymerase chain reaction (PCR) and standard DNA sequencing techniques, such as 16S ribosomal deoxyribonucleic acid (rDNA)-based denaturing gradient gel electrophoresis (DGGE) and terminal restriction fragment length polymorphism (T-RFLP) molecular fingerprinting methods, cloning, and more. The present invention is intended to encompass all DNA-based technologies routinely used to detect and identify microbes, based on the DNA sequence of those microbes, for the purpose of detecting, identifying, and quantifying microbes in a wastewater treatment facility.

PCR-based measurements, such as competitive PCR and competitive RT-PCR, can provide precise quantification of desired gene copies, or microbial count, is achieved, thereby providing information that can be used to predict more accurately the operational efficiency of a biotreatment system. Furthermore, direct information on the relative expression or activity of specific gene copies can be obtained for a system, and can be used to evaluate the capacity of a system to respond rapidly to changing environmental conditions.

After the DNA sequences have been ascertained, they can be analyzed to identify their microbial source. The sequences can be compared to the sequences of microbes that are stored on various bioinformatics platforms.

This can be currently facilitated by the Quantitative Insights Into Microbial Ecology (QIIME, www.qiime.org) open source software package, which is the most widely used software for the analysis of microbial community data generated on high-throughput sequencing platforms. QIIME was initially designed to support the analysis of marker gene sequence data, but is also generally applicable to “comparative-omics” data (including but not limited to metabolomics, metatranscriptomics, and comparative human genomics).

QIIME is designed to take users from raw sequencing data (for example, as generated on the Illumina™ and 454™ platforms) though the processing steps mentioned above, leading to quality statistics and visualizations used for interpretation of the data. Because QIIME scales to billions of sequences and runs on systems ranging from laptops to high-performance computer clusters, it is expected that it or similar systems will continue to keep pace with advances in sequencing technologies to facilitate characterization of microbial community patterns ranging from normal variations to pathological disturbances in many human, animal and environmental ecosystems.

For microbiome data analysis, the following steps will be taken. Unless otherwise noted, the steps will be performed with QIIME. However, other such systems can be used and the scope of protection afforded to embodiments is not in any way limited to, or dependent upon, the use of QIIME.

A closed-reference taxonomic classification can be performed, where each sequence is aligned to the SILVA reference database, version 123. Sequences can be aligned using the UCLUST algorithm included in QIIME version 1.9.1. See Caporaso, J. G., Kuczynski, J., Stombaugh, J., Bittinger, K., Bushman, F. D., Costello, E. K., Knight, R. (2010), QIIME allows analysis of high-throughput community sequencing data, Nature Methods 7(5):335-336. A minimum threshold of 97% sequence identity was used to classify sequences according to representative sequences in the database. These sequences can then assigned a curated taxonomic label based on the seven level SILVA taxonomy.

This analysis preferably can be performed using tools such as QIIME Analysis Pipeline, Machine learning, and UniFrac. Also, Mothur is another open source software tool for analyzing the DNA from uncultured microbes. Both tools allow the identification and classification of microbes found in samples.

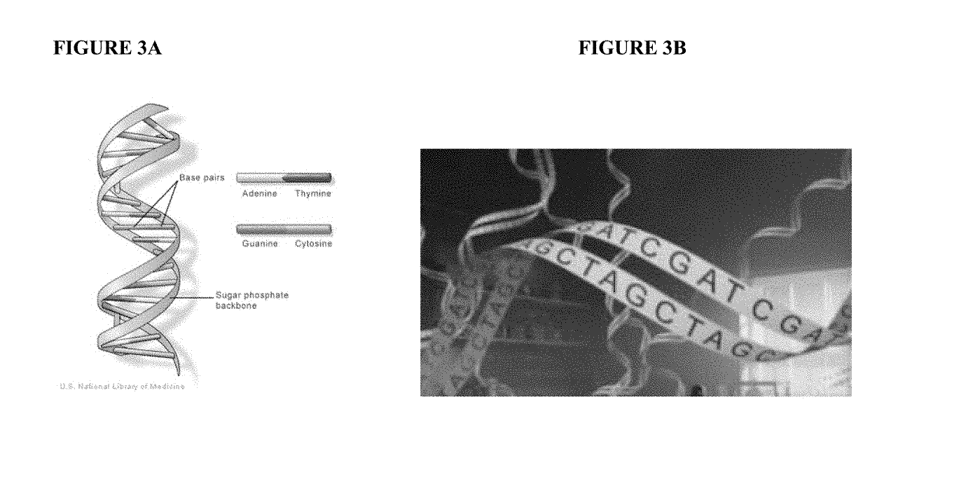

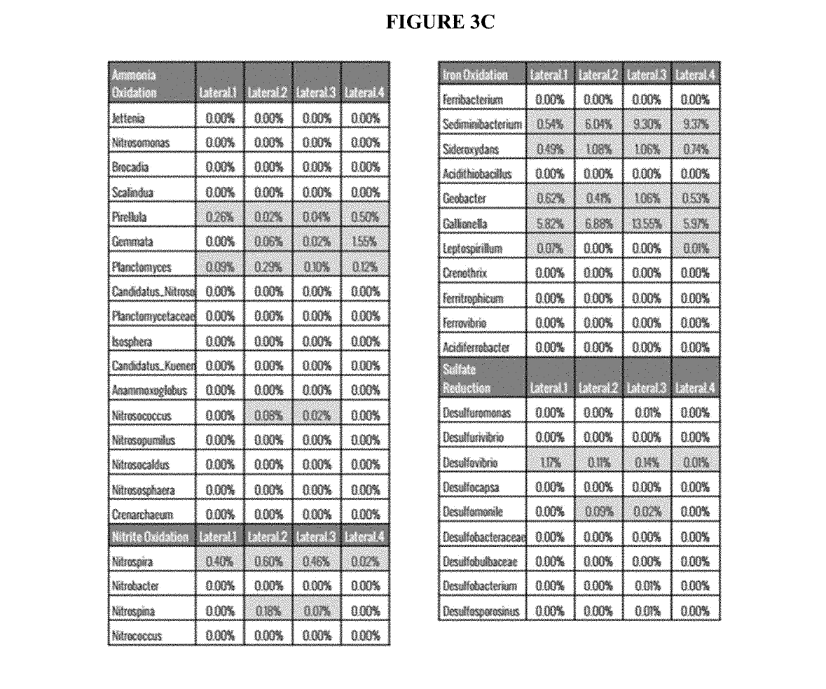

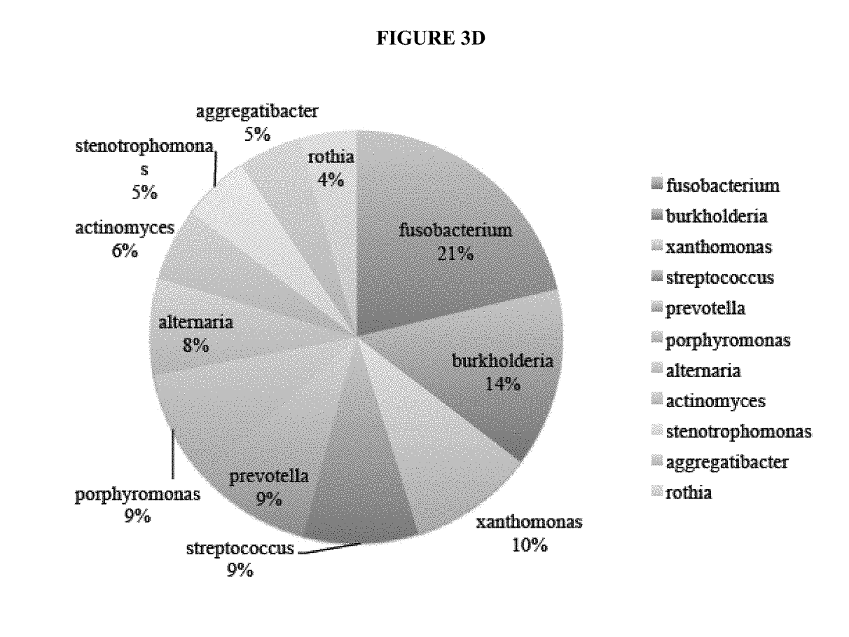

As shown in FIGS. 3C-3D, the DNA sequences can reveal the identity of the microbes found in a sample. In some cases, the relative proportions of each microbe (compared to the whole) can be determined. Further, this information can be aggregated to illuminate the relative proportions of certain types or families of bacteria, or to identify microbes with common functional features.

As shown in FIG. 3C, the presence of microbes specifically related to ammonia oxidation were detected in the samples. Here, Pirellula, Gemmata, and Planctomyces were found in detectable quantities, even though those quantities were less than 1% of the total microbe populations. Nitrosococcus was detected in two of the samples, but not detected in two others. Other microbes associated with ammonia oxidation, such as Jettenia, Nitrosomonas, Brocadia, and others, were not detected in any of the samples. This suggests that the samples contained a spectrum of microbes capable of ammonia oxidation.

Similarly, certain microbes capable of performing nitrite oxidation were found in the samples. Specifically, Nitrospira was identified in all of the samples; again, at less than 1% of the total microbial population. Nitrobacter and Nitrosococcus microbes were absent from all samples, while Nitrospino was detected in some of the samples, but not others.

Certain microbes relating to iron oxidation were detected in the samples, such as Sediminibacterium, Sideroxydans, Geobacter, and Gallionella in all samples in varying relative quantities, while Leptospirillum microbes were detected in half of the samples. Other functionally related microbes, such as Ferribacterium and Acidiferrobacter were absent from these samples.

Certain microbes relating to sulfate reduction were detected in the samples, such as Desulfovibrio in all samples in varying relative quantities, while Desulfomonile was detected in half of the samples. Many other functionally related microbes, such as Desulfosporosinus were absent from these samples.

The presence and relative quantities of a variety of microbes was determined. Further, by analyzing the microbes by function (for example, ammonia oxidation), the results indicate whether the sample possessed the ability to perform such functions in situ. By examining the results as groups of microbes with similar functions, it can be ascertained that while a particular microbe, such as Planctomyces, can constitute a small percentage of a total biomass, it can coexist with other microbes that perform the same function. This type of analysis shows the relative contribution of an individual species of microbe to the functionality of a biomass, as well as the total contributions of a group of microbes possessing similar functions.

This analysis indicates which beneficial microbes are present and can be preferentially cultured. It also provides candidate microbes for addition to the biomass, if the analysis shows that a particular functional feature is absent or insufficiently represented.

As shown in FIG. 3D, the relative proportions or percentages of different families or different types of microbes can be determined from these analyses. Fusobacterium (normally found in the human mouth and nasal cavities) make up 21% of this sample, while burkholderia (a pathogenic genus of microbes) make up 14% of this sample, and xanthomonas (which are found on many plants) make up 10% of this sample. Rothia microbes, which tend to reside in the mouth and respiratory tracts of different animals, make up 4% of this sample. These analyses show that many microbes from very different sources and having very different functions can be detected, identified, and quantified using the methods described.

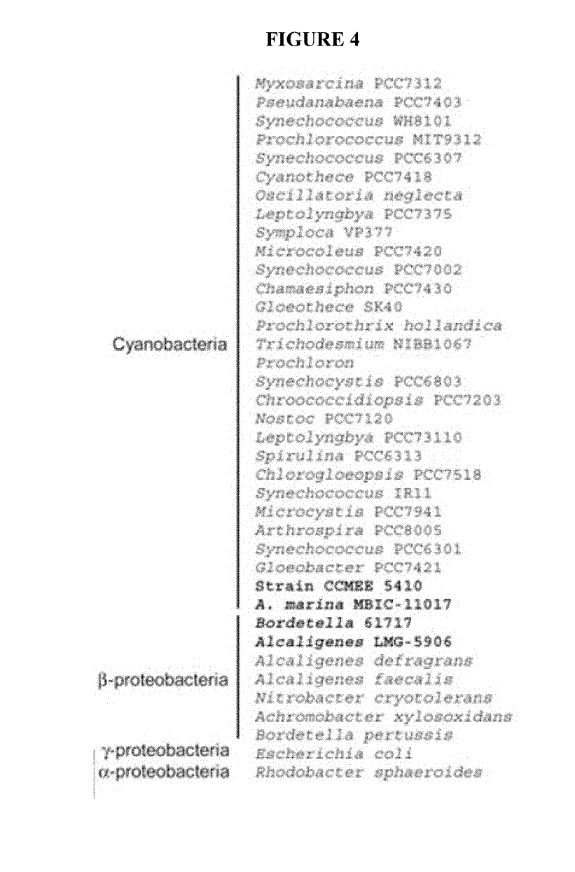

As shown in FIG. 4, families of microbes can be screened to identify microbes in a specific taxonomy (for example, cyanobacteria). Such searches can also be used to determine whether certain classes or types of bacteria are absent from a sample.

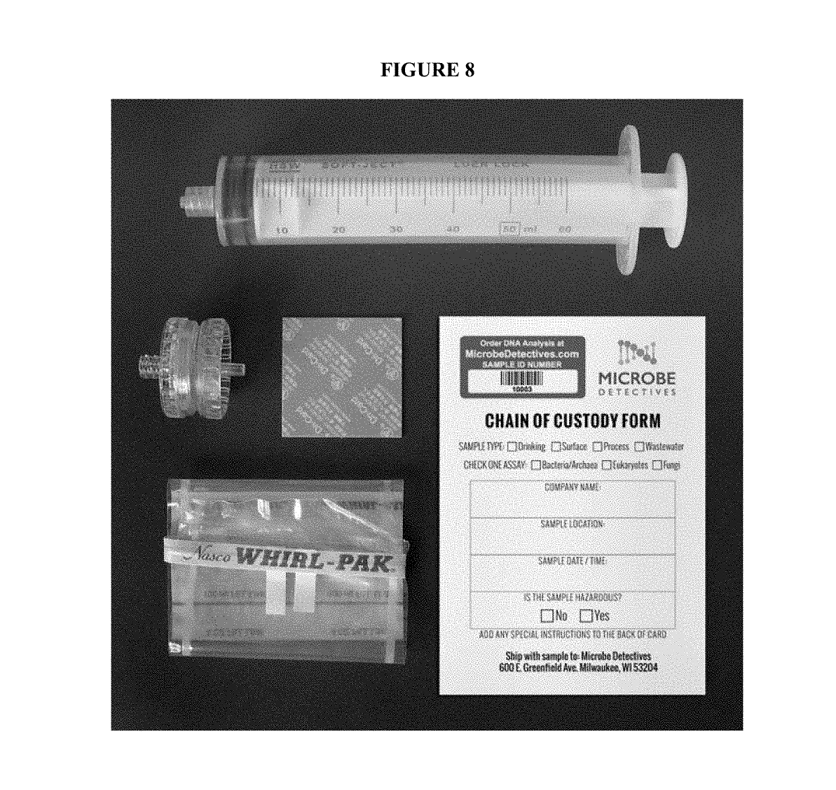

Referring now to FIG. 2 and FIG. 8, one aspect of an embodiment of the invention can include or be practiced in the form of a kit. Such a kit can include a means for collecting a liquid sample. For example, the sample can be collected in a container (preferably sterile or sterilized) such as a disposable lidded or capped cup, an Eppendorf tube, a conical centrifuge tube, or other collection container for storing liquid samples. The kit can also include a filter or other membrane or substrate for collecting bacterial or microbial samples. The kit can also include instructions for using the invention. The kit can also include a desiccant card (or other drying material) for keeping collected samples dry during storage or shipping.

As shown in FIGS. 2 and 8, the kit can include a syringe (for example, a plastic syringe) with filter unit for attaching to the end of the syringe. The filter unit is preferably sterile and has a filter housing surrounding the filter, to avoid contamination when handled by the user.

It is envisioned that a user will collect a sample in a clean vessel or container, the sample being well-mixed. The syringe can be used to draw water or liquid from the collection vessel into the syringe. The attachable filter (for example, a filter protected within a filter housing) can be attached to an end of the syringe, and the sample can be expelled from the syringe and through the filter. The water or liquid is expelled from the syringe, while the microbes are trapped onto the filter. The filter unit can be removed. This procedure can be repeated to sample large volumes or liquid.

The filter can be immediately subjected to DNA extraction, amplification, and identification, or can be dried or stored or shipped to a remote location prior to those subsequent steps.

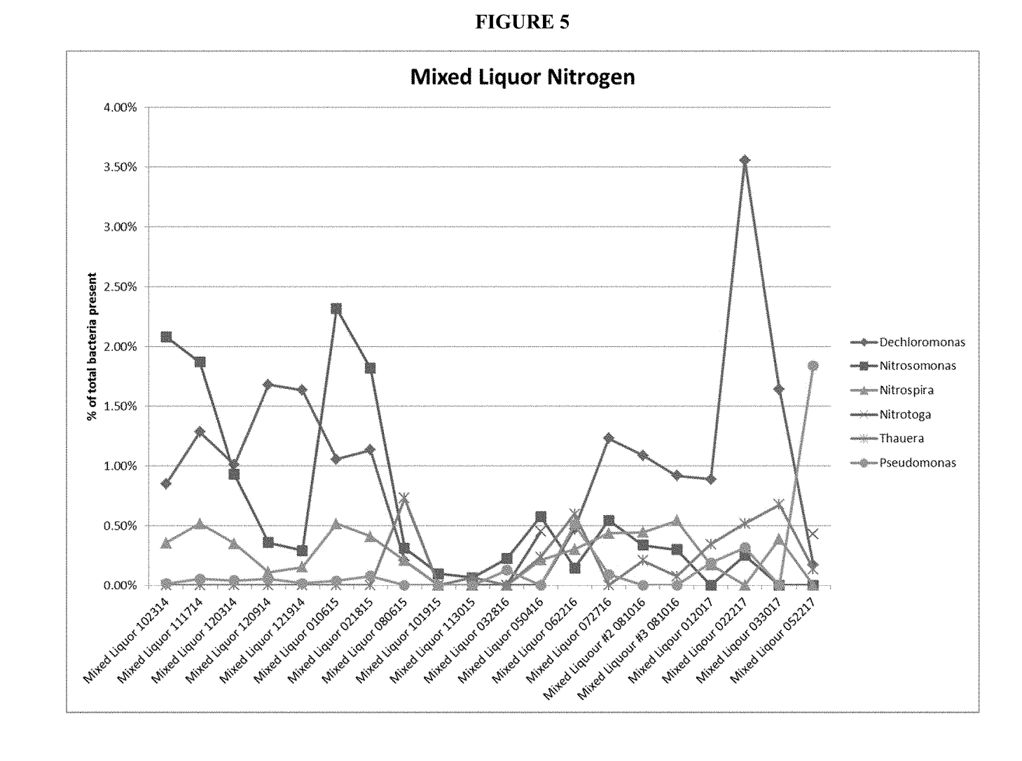

As shown in FIGS. 5-7, analyses are performed on samples collected from water systems or wastewater treatment systems. As shown in FIG. 5, mixed liquor samples are collected at various time points, over the course of several years. The samples are processed according to the disclosed invention, and various bacteria are detected, identified, and quantified.

In this figure, an analysis is performed on microbes relating to the removal of organic nitrogen from wastewater: Dechloromonas, Nitrosomonas, Nitrospira, Nitrotoga, Thauera, and Pseudomonas.

Initially, Dechloromonas, Nitrosomonas, Nitrospira, Nitrotoga are present as nitrogen-processing microbes, but later fall to nearly zero levels during the timeline. The Dechloromonas microbes return to having similar levels or percentages, while the expression of Nitrosomonas, Nitrospira, Nitrotoga, and Thauera microbes remain lower than their initial levels or percentages, and while the presence of Pseudomonas microbes changed from minimal to the second-most-abundant group in the analysis.

The disclosed invention allows the monitoring of a system to track the growth and death of individual microbe species or microbe families over time.

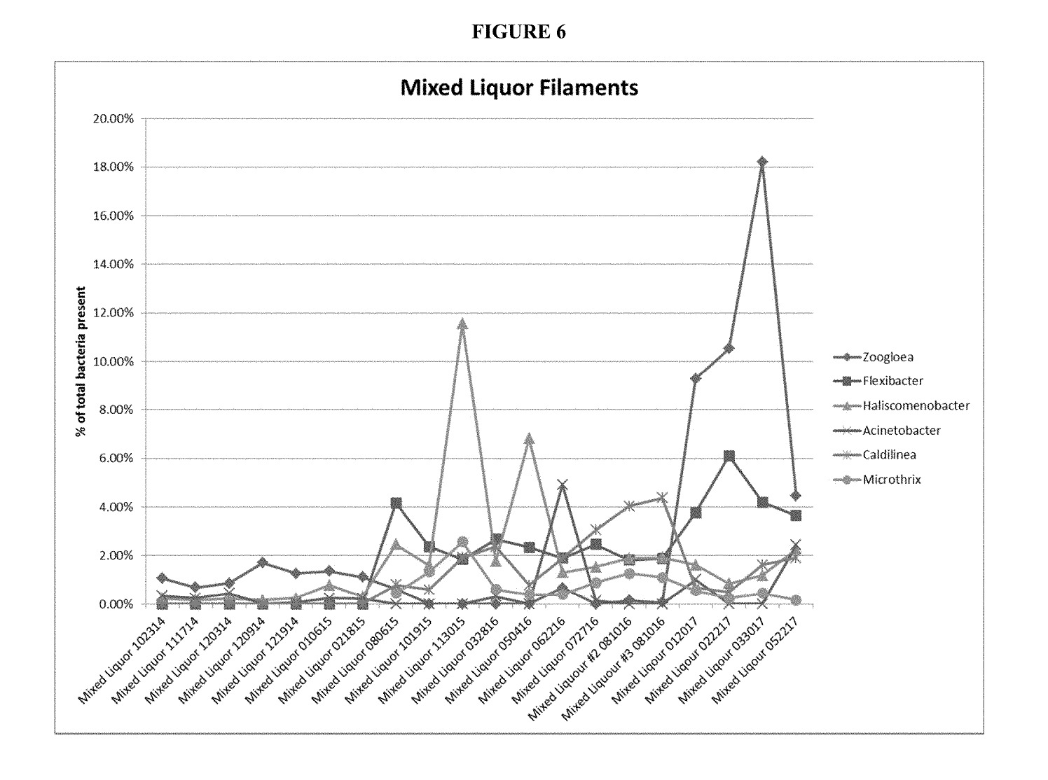

As shown in FIG. 6, analyses similar to those shown in FIG. 5 are performed, but with the analysis focusing on the presence and amount of filamentous microbes present in the sample over time. Here, Zoogloea microbes show a steady presence in the total bacterial pool, hovering at about 1% of the microbes, until a later time, when the Zoogloea comes to represent a peak of 18% of the total bacteria. This suggests a sudden growth of these microbes, or a sudden death of other microbes in comparison. Flexibacter microbes appears undetectable until midway through the analysis, then establishes itself as 2-6% of the bacterial population over the latter portion of the timeline.

Similarly, other microbes exhibit trends as increasingly greater percentages of the bacterial pool (such as Haliscomenobacter) or show temporary periods as comprising a greater percentage of the total bacterial pool (such as Caldilinea).

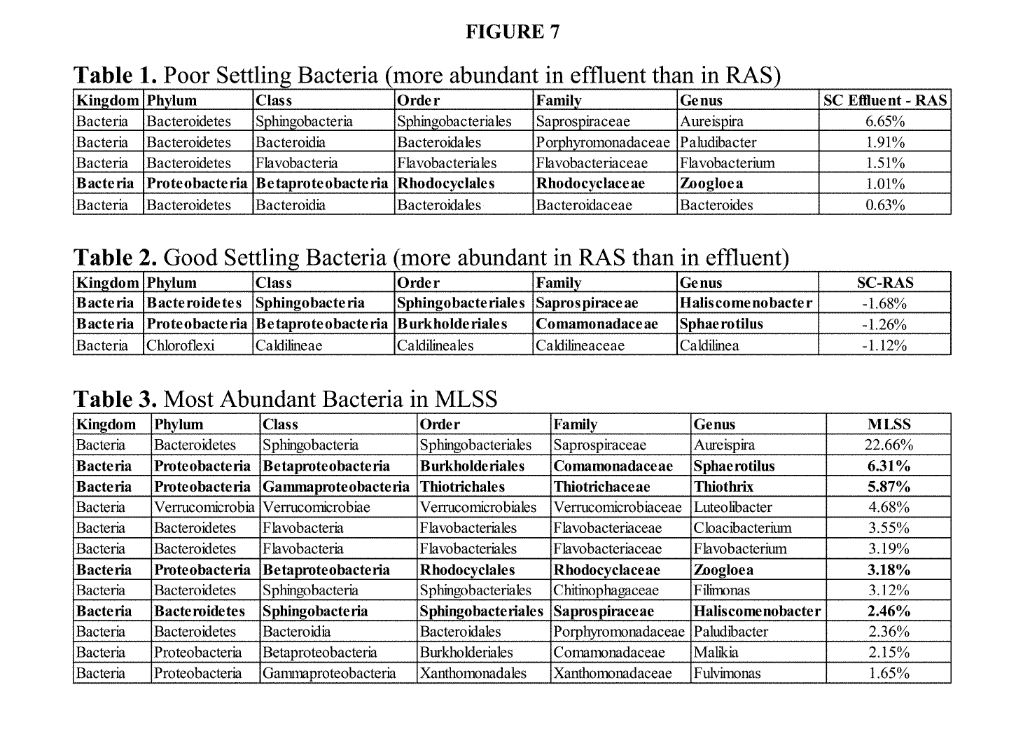

As shown in FIG. 7, the presence of different specific microbes at different locations in a water treatment system are listed. The presence of bacteria considered to be poor for settling conditions are found in greater abundance in the effluent exiting the system than in the RAS portion of the system. The presence of bacteria considered to be good for settling are found in greater quantities in the RAS than in the effluent.

Further, specific bacteria are found in the MLSS and ranked by their relative abundance within that site.

The analyses show that where certain of the detected microbes possessed similar functions, the functionality could be produced by microbes that were closely related or by microbes that were very distantly related. For example, most of the bacteria described as “Poorly Settling Bacteria” belonged to the same phylum (Bacteroidetes), but one of the microbes belonged to a different Phylum (Proteobacteria).

An embodiment of the invention could be used to determine a course of action to identify strategies for analyzing the performance of a wastewater facility, identify the group of microbes contained in the biomass (and analyze the ability of the biomass to perform specific biological processes on the wastewater), and to optimize the composition of the biomass. An embodiment of the invention could also be used to monitor the response of the biomass toward external environmental stressors and toward efforts to maintain or improve the biomass. An embodiment of the invention could also be used to detect the incursion of undesirable microbes that are indicative of contamination, could impair the functionality of the biomass, or could cause disease.

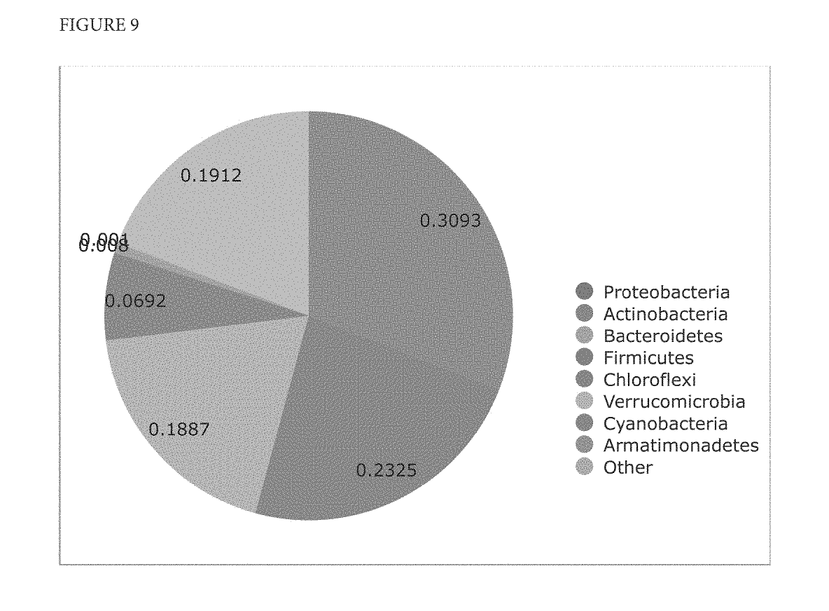

Referring now to FIG. 9 there is shown a pie chart depicting that Proteobacteria 30.93%, Actinobacteria 23.25%, Bacteroidetes 18.87%, Firmicutes 6.92%, Chloroflexi 0.8%, Verrucomicrobia 0.1% and Cyanobacteria 0% are detected in this sample.

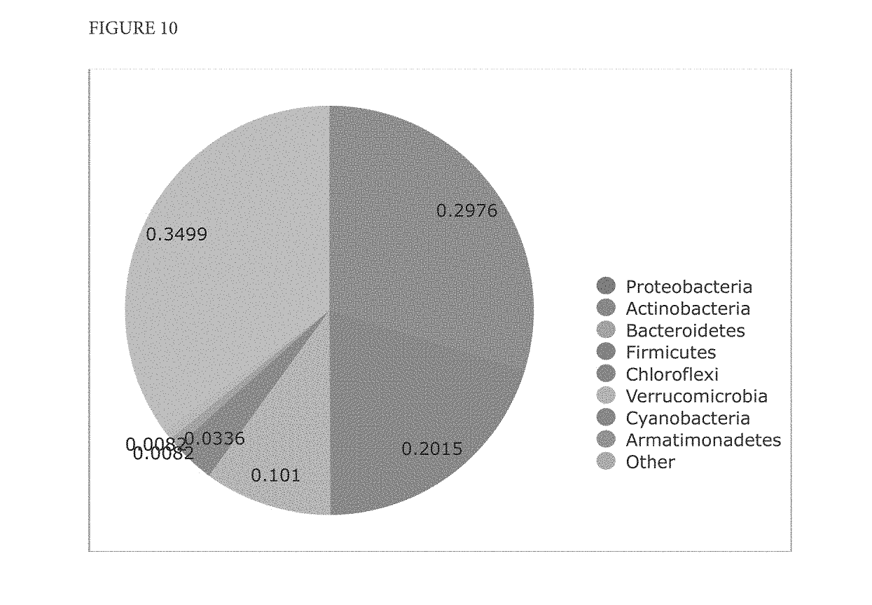

Referring now to FIG. 10 there is shown a pie chart depicting that Proteobacteria 29.76%, Actinobacteria 20.15%, Bacteroidetes 10.1%, Firmicutes 3.36%, Chloroflexi 0.82%, Verrucomicrobia 0.82% and Cyanobacteria 0% are detected in this sample.

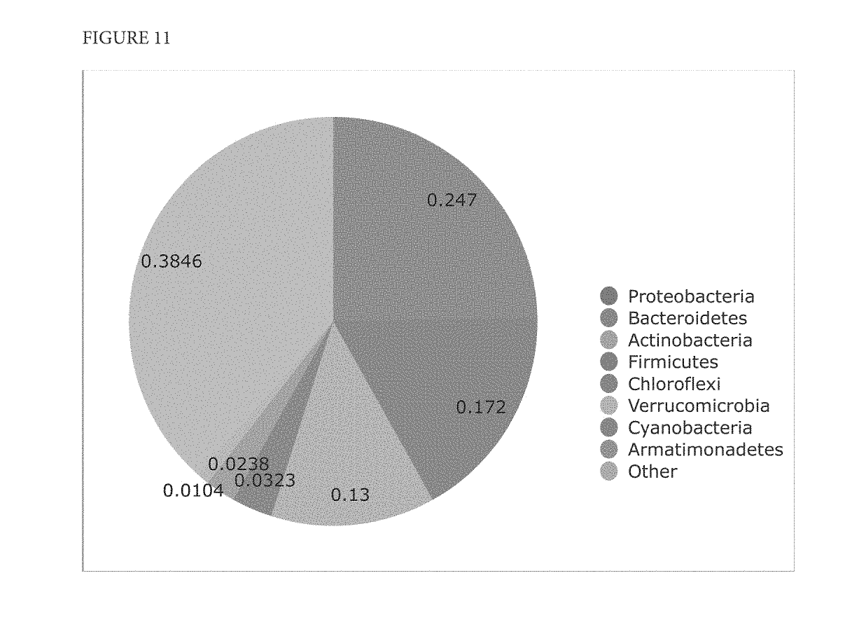

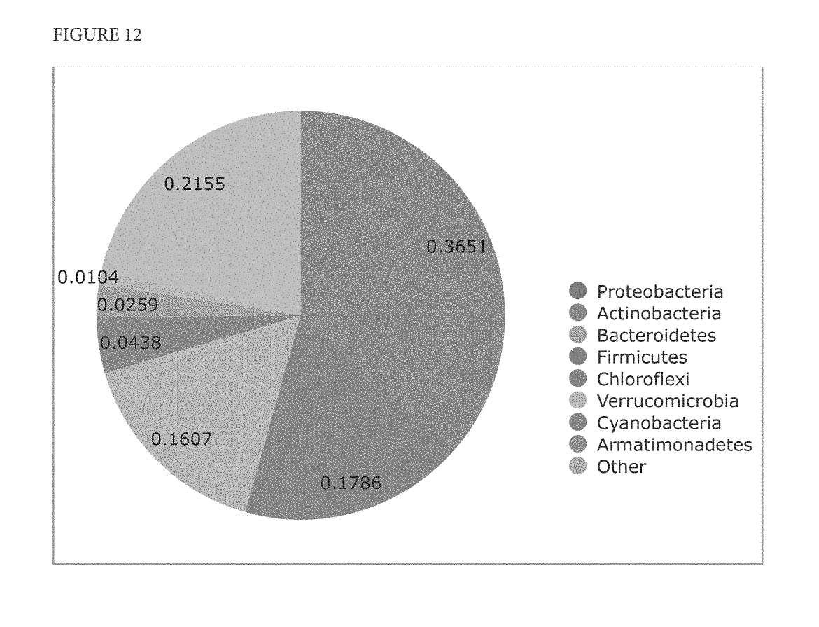

Referring now to FIG. 11 there is shown a pie chart depicting that Proteobacteria 24.7%, Bacteroidetes 17.2%, Actinobacteria 13%, Firmicutes 3.23%, Chloroflexi 2.38%, Verrucomicrobia 1.04% and Cyanobacteria 0% are detected in this sample.

Referring now to FIG. 12 there is shown a pie chart depicting that Proteobacteria 36.51%, Actinobacteria 17.86%, Bacteroidetes 16.07%, Firmicutes 4.38%, Chloroflexi 2.59%, Verrucomicrobia 1.04% and Cyanobacteria 0% are detected in this sample.

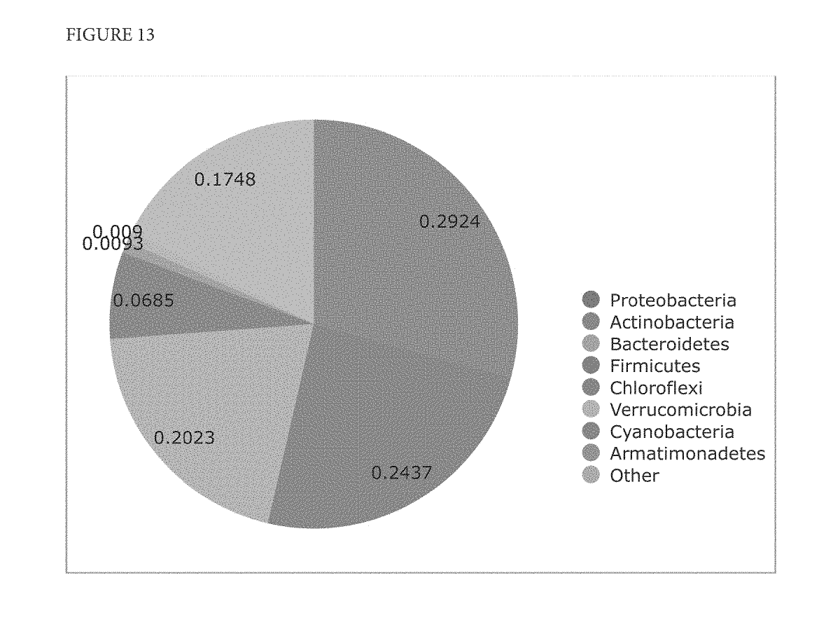

Referring now to FIG. 13 there is shown a pie chart depicting that Proteobacteria 29.24%, Actinobacteria 24.37%, Bacteroidetes 20.23%, Firmicutes 6.85%, Chloroflexi 0.93%, Verrucomicrobia 0.9% and Cyanobacteria 0% were detected in this sample.

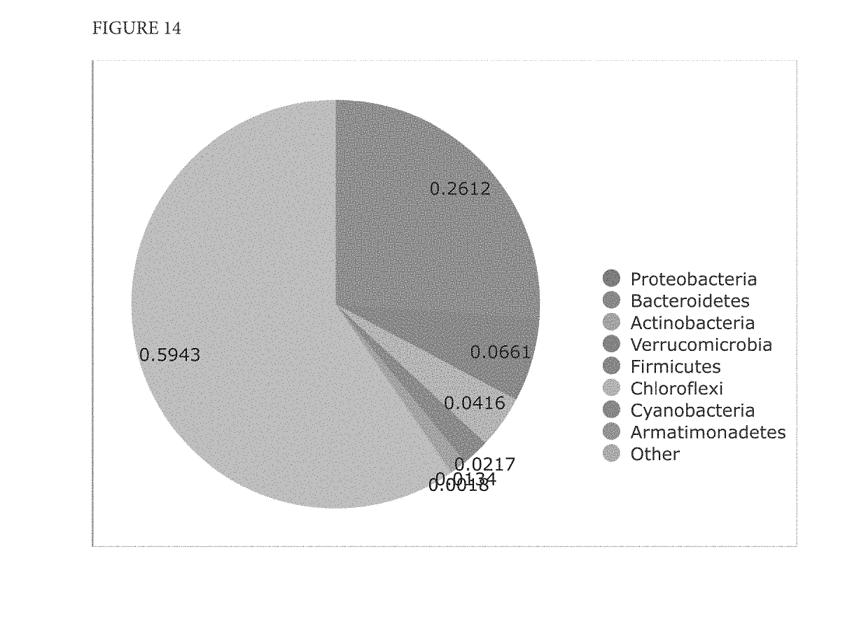

Referring now to FIG. 14 there is shown a pie chart depicting that Proteobacteria 26.12%, Bacteroidetes 6.61%, Actinobacteria 4.15%, Verrucomicrobia 2.17%, Firmicutes 1.34%, Chloroflexi 0.18% and Cyanobacteria 0% are detected in this sample.

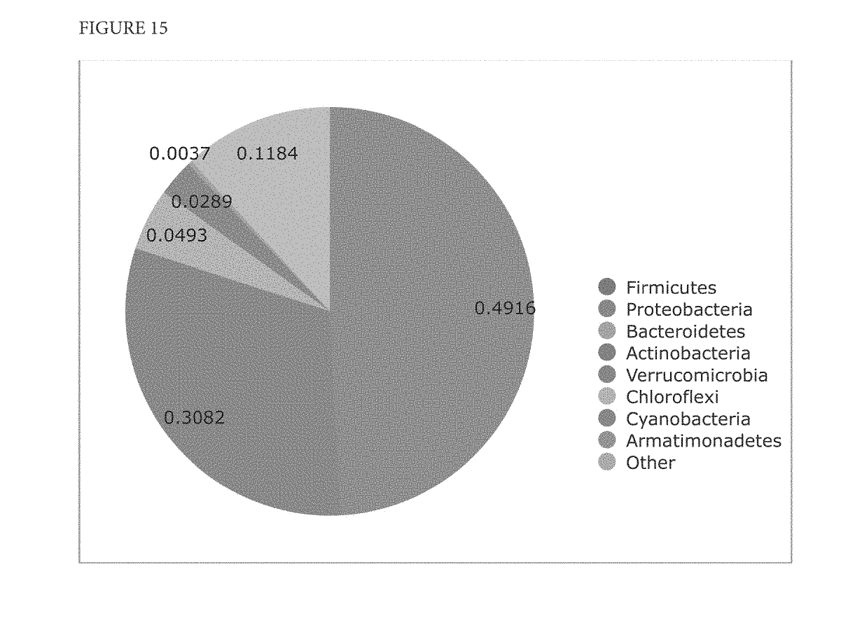

Referring now to FIG. 15 there is shown a pie chart depicting that Firmicutes 49.16%, Proteobacteria 30.82%, Bacteroidetes 4.93%, Actinobacteria 2.89%, Verrucomicrobia 0.37% and Chloroflexi 0% are detected in this sample.

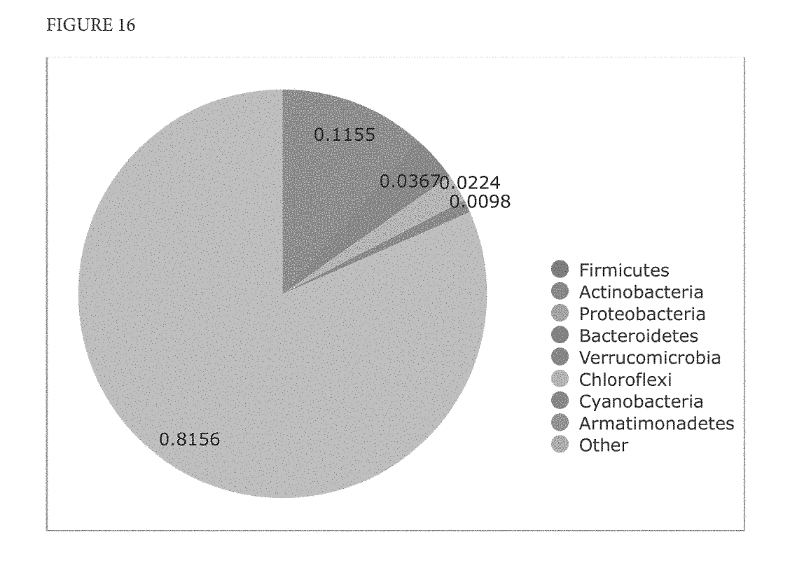

Referring now to FIG. 16 there is shown a pie chart depicting that Firmicutes 11.55%, Actinobacteria 3.67%, Proteobacteria 2.24%, Bacteroidetes 0.98% and Verrucomicrobia 0% are detected in this sample.

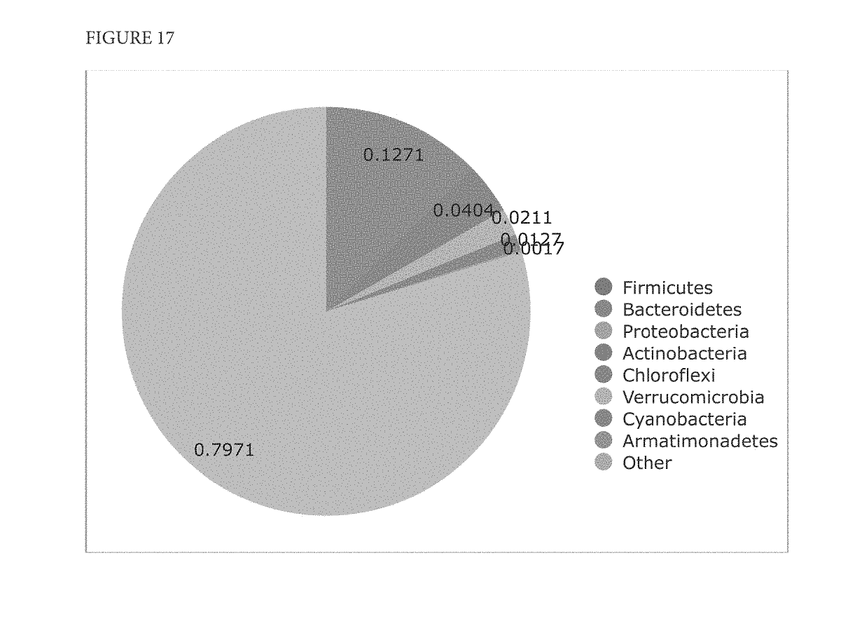

Referring now to FIG. 17 there is shown a pie chart depicting that Firmicutes 12.71%, Bacteroidetes 4.04%, Proteobacteria 2.11%, Actinobacteria 1.27%, Chloroflexi 0.16% and Verrucomicrobia 0% were detected in this sample.

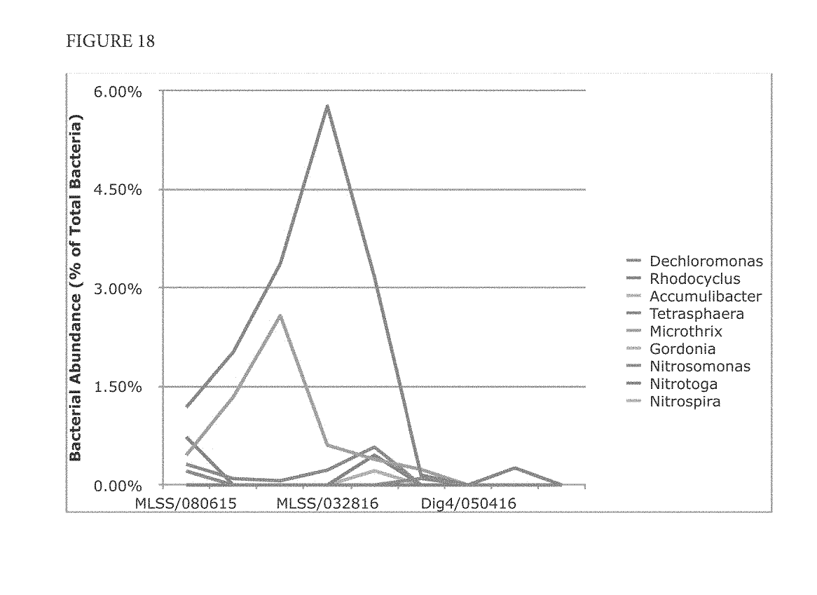

Referring now to FIG. 18 there is shown a bacterial abundance chart depicting changes in bacterial abundance across different samples.

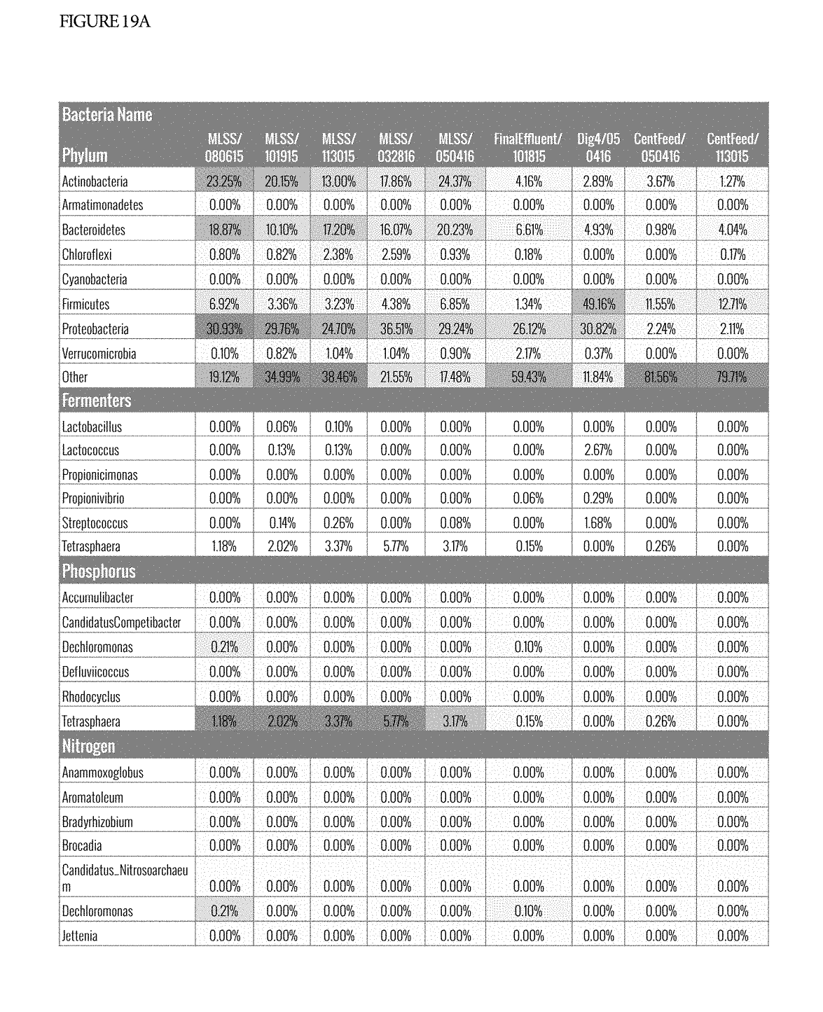

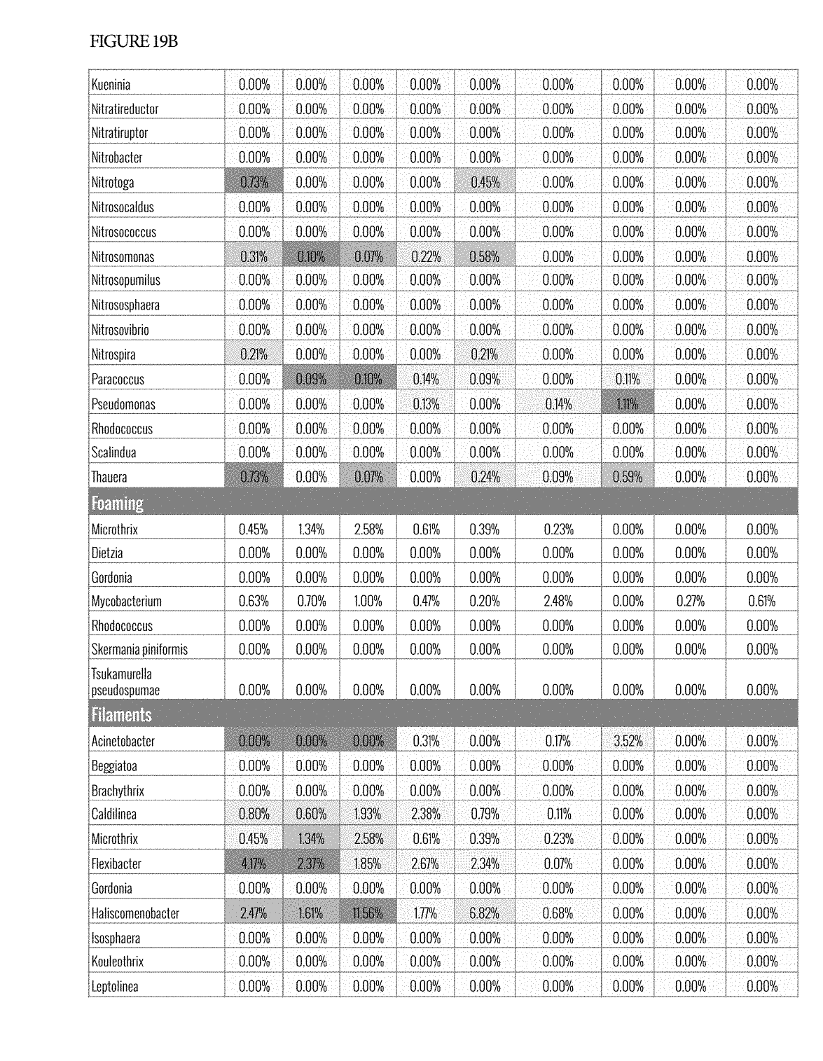

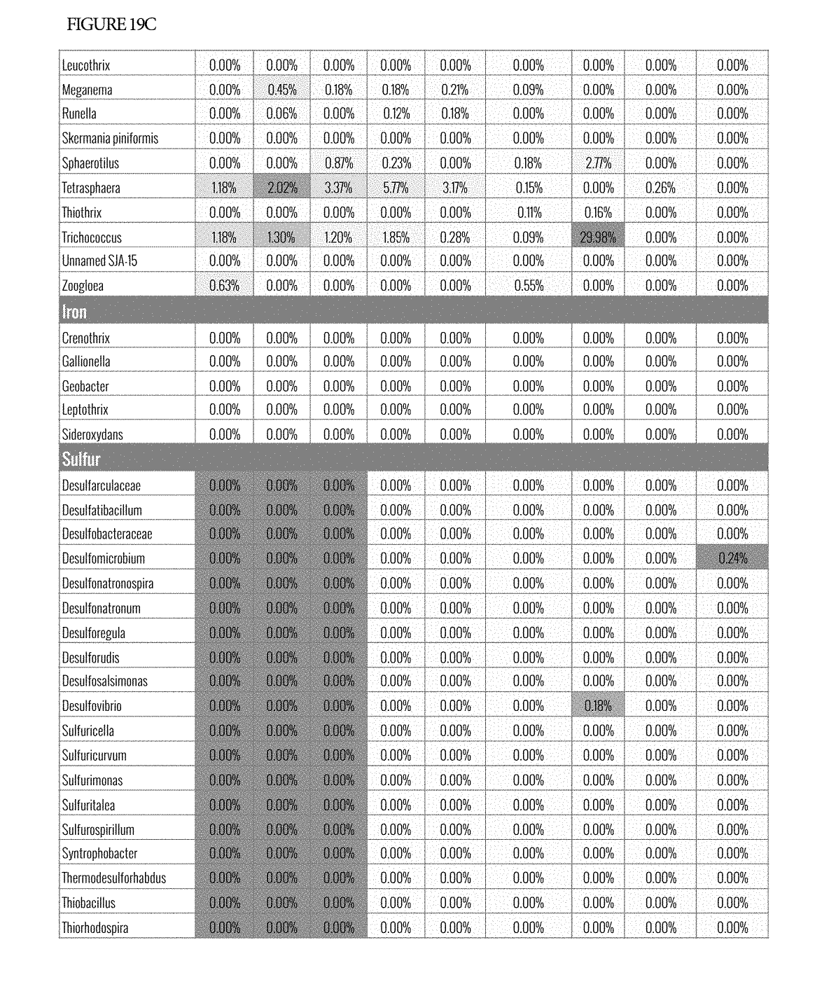

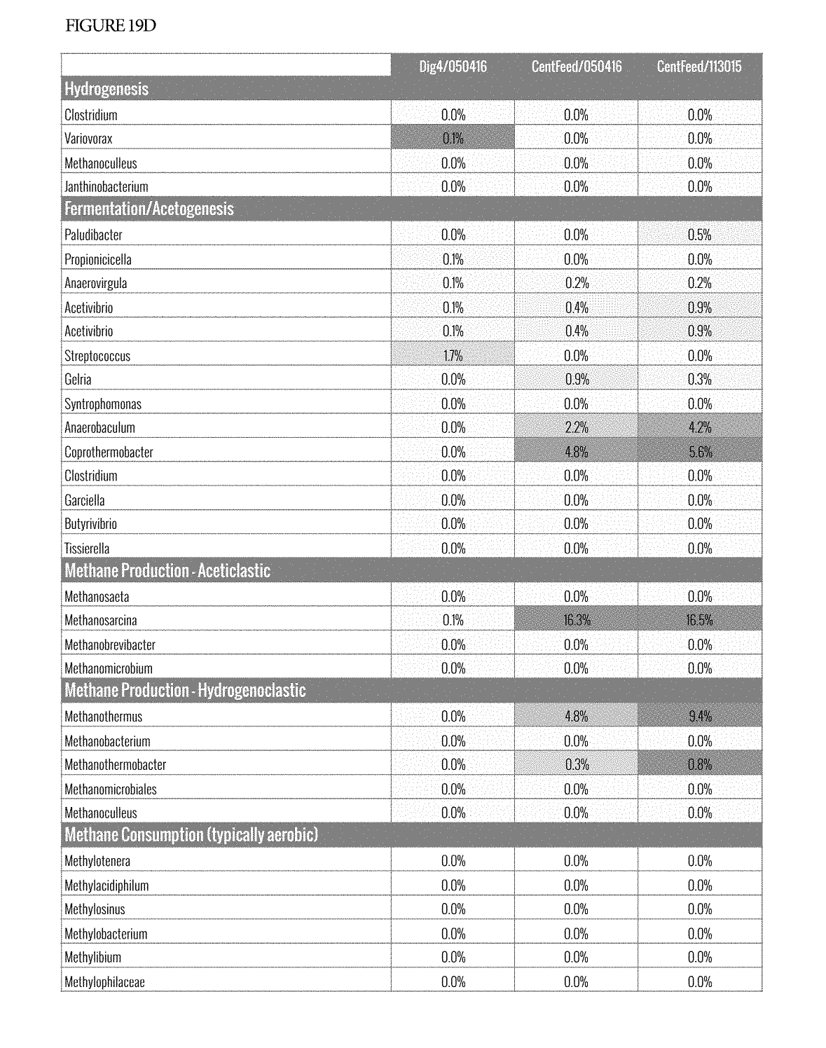

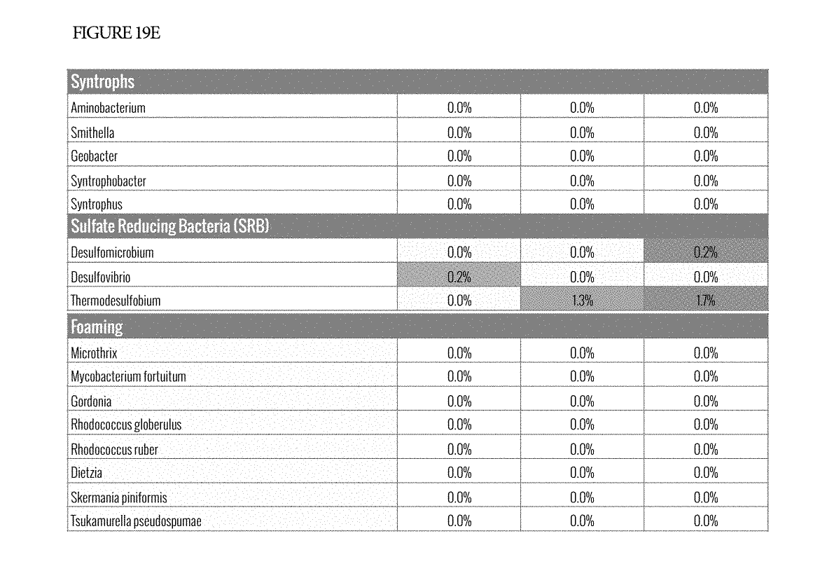

Referring now to FIGS. 19A-E, there is shown a bacterial relative abundance table, illustrating how bacteria can be identified in multiple samples and their relative abundance compared as between samples.

It should be understood, of course, that the foregoing relates to exemplary embodiments of the invention and that modifications can be made without departing from the spirit and scope of the invention as set forth in the following claims

Embodiments of the present invention are not limited to the particular details of the method/embodiment depicted, and other modifications and applications are contemplated. Certain other changes can be made in the above-described method without departing from the true spirit and scope of the invention herein involved. For example, the present method can be utilized with other types of liquid transport or storage systems, such as water fountains, closed buildings, pools, irrigation systems, waste treatment systems. It is intended, therefore, that the subject matter in the above depiction shall be interpreted as illustrative and not in a limiting sense.

What is claimed is:

1. A method of identifying a microbe in wastewater treatment, the method comprising:
receiving a sample taken from a sample point in a wastewater treatment system, the sample containing a microbe;
collecting a microbe from the sample, the microbe containing a microbial DNA sequence;
extracting the microbial DNA sequence, the microbial DNA sequence having a hypervariable region unique to the microbe;
amplifying a portion of the hypervariable region of the microbial DNA sequence;
determining the sequence of the amplified portion;
comparing the determined sequence of the amplified portion to a known DNA sequence unique to particular microbes; and
identifying from the comparing the microbe which the amplified portion was extracted based on the comparing step.

2. The method according to claim 1, in which the sample contains a plurality of microbes, further comprising identifying each of the plurality of microbes in the sample.

3. The method according to claim 2, further comprising determining the relative frequency of occurrence of each of member of the plurality of microbes within the sample.

4. The method according to claim 1, in which the wastewater treatment system comprises a biomass comprising microbes, the biomass being used in the water treatment system for the treatment of water.

5. The method according to claim 4, further comprising determining whether the microbe identified is a portion of the biomass used in the water treatment system for the treatment of water or a contaminant.

6. The method according to claim 1, further comprising taking multiple samples from one or more sample points at one or more points in time.

7. The method according to claim 1, further comprising comparison of the microbial content of the samples multiple samples from one or more sample points at one or more points in time.
